(19) United States
(12) Patent Application Publication
(10) Pub. No.: US 2026/0077335 A1
(43) Pub. Date: Mar. 19, 2026

(54) SQUARAMIDE COVALENT ORGANIC FRAMEWORKS

(71) Applicants: EXXONMOBIL TECHNOLOGY AND ENGINEERING COMPANY, Spring, TX (US); National University of Singapore, Singapore (SG)

(72) Inventors: Dan ZHAO, Singapore (SG); He LI, Singapore (SG); Lisa S. BAUGH, Ringoes, NJ (US)

(21) Appl. No.: 19/327,527

(22) Filed: Sep. 12, 2025

Related U.S. Application Data
(60) Provisional application No. 63/694,994, filed on Sep. 16, 2024.

(51) Int. Cl. B01J 20/26; B01D 53/02; B01J 20/30; C07D 259/00; C07D 487/22

(52) U.S. Cl. CPC B01J 20/262; B01D 53/02; B01J 20/3085; C07D 259/00; C07D 487/22; B01D 2253/202; B01D 2257/504

(57) ABSTRACT

Covalent organic framework compositions are provided that incorporate the nitrogen atoms of a squaramide functionality into the covalent organic framework structure while reducing or minimizing the number of nitrogen atoms in the minimum ring framework path of the covalent organic framework. Methods for forming such covalent organic framework compositions are also provided.

### CROSS-REFERENCE TO RELATED APPLICATIONS AND PRIORITY CLAIM

This non-provisional patent application claims priority to U.S. provisional patent app. No. 63/694,994, filed Sep. 16, 2024, and titled “Squaramide Covalent Organic Frameworks,” the entire contents of which is incorporated herein by reference.

### FIELD OF THE INVENTION

Covalent organic framework compositions that incorporate squaramide functionality, and corresponding methods of making such compositions, are provided.

### BACKGROUND OF THE INVENTION

Covalent organic framework (COF) materials are a relatively new class of materials. Some COFs have potential application as sorbents for separations.

Some types of COFs that have potential application for separation of CO₂ are COFs that incorporate a squaramide functionality as part of the framework. Squaramide is a derivative of squaric acid, where each —OH groups in the acid functionalities is replaced with either a primary amine (—NH₂) or a secondary amine (—NHR). The simplest substitution is to replace both of the —OH groups in the acid functionalities with —NH₂ to make a primary amine, which corresponds to a squaramide molecule. More generally, the —OH groups can be replaced with —NHR, where R can be hydrogen (to make a primary amine), or R can be some type of organic group where R is connected to the nitrogen via a nitrogen-carbon bond, thus forming a secondary amine.

Due to the constrained nature of the 4-membered ring, and the resulting proximity of the two squaramide amine groups, compounds (such as covalent organic frameworks) that incorporate squaramide functionality have the potential to provide desirable functionality for CO₂ adsorption.

Some COFs incorporating squaramide functionality into a covalent organic framework have been previously reported. Examples of such references include 1) Li et al., Chem. Commun. Vol. 55, pg 5423 (2019); 2) Huang et al., Angew. Chem. Int. Ed. Engi. Vol. 61 c202206749 (2022); 3) Shi et al., Adv. Sci. Vol. 10, 2302925 (2023); and 4) Kushwaha et al., Adv. Energy Mater. Vol. 13, 2301049 (2023).

An example of a procedure for making a squaric acid methyl ether is described in Claveau et al. Chem Commun. 2018, 54, 3231. An example of a procedure for making 1,3,6,8-tetrakis(4-aminophenyl-pyrene), Py-NH₂ is described in Ascherl et al. J Am Chem Soc, 2019, 141, 15693. An example of a procedure for making 2,4,6-tris(4-aminophenyl)-1,3,5-triazine (TAPT) is described in Zhang et al. Angew. Chem. Int. Ed. Engl. 2018, 57, 16754. An example of a procedure for making 4,4′,4″-(1,3,5-triazine-2,4,6-triyl)tris [(1,1′-biphenyl)-4-amine], TTBA, is described in Dey et al. J. Am. Chem. Soc. 2017, 139, 13083.

### SUMMARY OF THE INVENTION

In an embodiment, a method for forming a covalent organic framework composition that incorporates squaramide functionality is provided. The method includes reacting a squaric acid alkyl ether with at least one amine-containing reagent in the presence of an organic strong acid under framework synthesis conditions to form a covalent organic framework having squaramide functional groups, wherein nitrogen atoms of the squaramide functional groups in the covalent organic framework correspond to 51% or more of nitrogen atoms in the minimum ring framework path of a ring defined by the covalent organic framework. The at least one amine-containing reagent has a central core and a plurality of amine-containing branches, the plurality of branches corresponding to 3 branches or 4 branches. Each branch in the plurality of amine-containing branches contains a primary amine and at least one aromatic ring. The central core comprises one or more additional aromatic rings, a plurality of fused aliphatic rings, a branch point central carbon, a branch point central nitrogen, or a combination thereof. The ether groups of the squaric acid alkyl ether each contain 1 to 4 carbon atoms.

In another embodiment, a covalent organic framework composition that incorporates squaramide functionality is provided. The covalent organic framework composition contains squaramide functional groups. The nitrogen atoms of the squaramide functional groups in the covalent organic framework material correspond to 51% or more of nitrogen atoms in the minimum ring framework path of a ring defined by the covalent organic framework. The minimum ring framework path further incorporates atoms from a plurality of aromatic rings, a number of rings in the plurality of aromatic rings being equal to or greater than a number of nitrogen atoms of squaramide functional groups in the minimum ring framework path. In some embodiments, the minimum ring framework path of a ring defined by the covalent organic framework contains 44 heavy atoms to 170 heavy atoms, while in other embodiments the minimum ring framework path of a ring defined by the covalent organic framework contains 66 heavy atoms to 210 heavy atoms.

### BRIEF DESCRIPTION OF THE DRAWINGS

FIG. 1 shows an example of a conventional covalent organic framework structure.

FIG. 2 shows an example of a reaction for forming a covalent organic framework that includes squaramide functionality.

FIG. 3 shows an example of a reaction for forming a covalent organic framework that includes squaramide functionality.

FIG. 4 shows an example of a reaction for forming a covalent organic framework that includes squaramide functionality.

FIG. 5 shows XRD spectra and TEM images for various covalent organic frameworks.

FIG. 6 shows FT-IR data for a covalent organic framework referred to as NUS-17.

FIG. 7 shows FT-IR data for a covalent organic framework referred to as NUS-18.

FIG. 8 shows FT-IR data for a covalent organic framework referred to as NUS-19.

FIG. 9 shows ¹³C NMR spectra for various covalent organic frameworks.

FIG. 10 shows an N₂ adsorption isotherm for a covalent organic framework referred to as NUS-17.

FIG. 11 shows an N₂ adsorption isotherm for a covalent organic framework referred to as NUS-18.

FIG. 12 shows an N₂ adsorption isotherm for a covalent organic framework referred to as NUS-19.

FIG. 13 shows CO₂ breakthrough sorption data (15% CO₂ in nitrogen at varying relative humidity) for NUS-17.

FIG. 14 shows CO₂ breakthrough sorption data (15% CO₂ in nitrogen at varying relative humidity) for NUS-18.

FIG. 15 shows CO₂ breakthrough sorption data (15% CO₂ in nitrogen at varying relative humidity) for NUS-19.

### DETAILED DESCRIPTION

All numerical values within the detailed description and the claims herein are modified by “about” or “approximately” the indicated value, and take into account experimental error and variations that would be expected by a person having ordinary skill in the art.

### Overview

In various aspects, covalent organic framework compositions are provided that incorporate the nitrogen atoms of a squaramide functionality into the covalent organic framework structure while reducing or minimizing the number of nitrogen atoms in the minimum ring framework path of the covalent organic framework. Methods for forming such covalent organic framework compositions are also provided.

Covalent organic frameworks (COFs) correspond to organic polymeric materials that are typically formed by bonding together a plurality of smaller poly-functional organic compounds, or secondary building units, having reactive groups with suitable geometric orientation to form ordered framework structures with defined porosity. A covalent organic framework can exist as a two-dimensional layered structure or a three-dimensional structure. For some types of COFs that correspond to two-dimensional layered structures, it may be possible to assemble the COF in a manner so that the structural features of the layers are not precisely aligned between layers (eclipsed model vs. staggered model), in a manner that reduces or minimizes the pore structures.

In a COF material, the bonding of the secondary building units is facilitated by the presence of heteroatoms different from carbon and hydrogen within the secondary building units. As an example, some types of COFs can form by condensation reactions where a di-aldehyde compound links or bonds together other secondary building units that include amines. This results in formation of imine bonds at the locations where the remaining portion of the di-aldehyde molecule connects to the other secondary building units. More generally, various types of covalent organic framework materials can include imine functional groups as part of the COF. Such imine groups can be part of the framework structure of the COFs, pendant groups or side chains attached to the framework structure of the COF, and/or any other location in the COF. Still more generally, COFs can be formed that include various types of functional groups that form part of the framework structure of the COF and/or that correspond to side chains attached to the framework structure of the COF.

For applications related to CO₂ adsorption, a beneficial type of functional group to have in a COF is an amine functionality, such as the nitrogen present in aliphatic amines. Aliphatic amines are favorable sites for CO₂ adsorption, but their strong nucleophilicity is not compatible with the typical chemistry used to make imine-linked COFs via condensation (reaction between an aldehyde and an amine) since they tend to react in the condensation step. The exact sorption properties of an amine site can vary based on a wide variety of factors. The development of new functional linkages in COFs that are beneficial for CO₂ sorption remains an underexplored area of research.

Amine sites in a COF are in contrast to imine sites. Although imines in a COF also involve nitrogen-carbon bonds, an imine in a COF framework involves a double bond between the nitrogen and one carbon and a single bond between the nitrogen and another carbon. On average, such imine sites have a substantially lower potential to serve as CO₂ sorption sites as compared with amine linkages (which are connected to neighboring carbons by only single bonds).

Squaramide is a four-member ring structure that includes a carbon-carbon double bond bearing amine substituents at each terminus of the double bond, along with carbonyl substitution at the two remaining ring carbons. The structure of squaramide is shown in Formula I. Although this molecule and its derivatives are referred to as “squaramides,” they are not amides. An amide functionality features a single bond between a carbon and a nitrogen originating on a carbon atom that also bears a carbonyl C═O double bond to oxygen. An amide group can be represented as —C(═O)NR₂ wherein the two R groups can be the same or different, and correspond to hydrogen or carbon-based groups such as alkyl or aryl. In squaramide and its derivatives, the carbonyl C═O bond and the C—N amine bond originate on different carbon atoms. Squaramide contains two amine groups in close proximity attached to a four-membered cyclobutene ring, which constrains the local geometry of these amine groups and creates special resonance and other hydrogen bonding effects among the components and substituents of the ring. The unusual resonance, olefinic bonding, and geometric distortion present in the squaramide structure result in greater electronic communication between the carbonyl and amine groups than would be otherwise typical. This feature has resulted in the common name, “squaramide.”

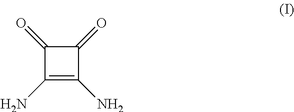

In addition to being a potential source of amine functionality in a COF material, squaramide functional groups provide an ability to incorporate two amine functional groups in a COF material that are relatively close together. Without being bound by any particular theory, it is believed that the close proximity of amine groups that is provided by a squaramide functional group can provide for beneficial adsorption properties for CO₂. It is also believed that the carbonyl groups of the squaramide may provide beneficial adsorption properties through their ability to participate in hydrogen bonding with water (a co-reactant in the formation of ammonium bicarbonate amine —CO₂ sorption products), and their role in creating polarization in the squaramide group, which may promote reaction or assist in the stabilization of sorption products.

Conventionally, a typical pathway for COF synthesis is to use one reagent that includes a plurality of terminal (primary) amine groups, along with a preformed internal squaramide group, and a second reagent that includes a plurality of aldehyde groups. For example, the first reagent can include two primary amine groups and an internal squaramide group while the second reagent contains three aldehyde groups. When these two reagents are condensed together, imines are formed, with the release of water. Conventional methods for synthesizing a COF that contains squaramide functional groups have used preformed squaramide functionalities substituted with extending groups containing terminal aromatic amines. In this type of synthesis pathway, the squaramide is first reacted with another reagent to form a larger compound having two terminal primary amine groups, where the nitrogens from the squaramide reagent become internal secondary amines in the interior of the larger compound. These secondary amines are not reactive under the COF synthesis conditions of imine formation, which allows the squaramide functionality to remain present in the COF after synthesis. Typically, the extending groups added to the squaramide reagent are aromatic rings bearing primary amine substituents in the para position to the squaramide. The primary amines present at the termini of the extending groups have typical geometric and electronic properties and react in a traditional COF condensation synthesis with a reagent such as a multi-aldehyde, forming imines (through conventionally known Schiff's base reactions for COF construction). These imine sites, specifically their nitrogen atoms, have a substantially lower potential to serve as CO₂ sorption sites as compared with amine sites such as those present in the squaramide.

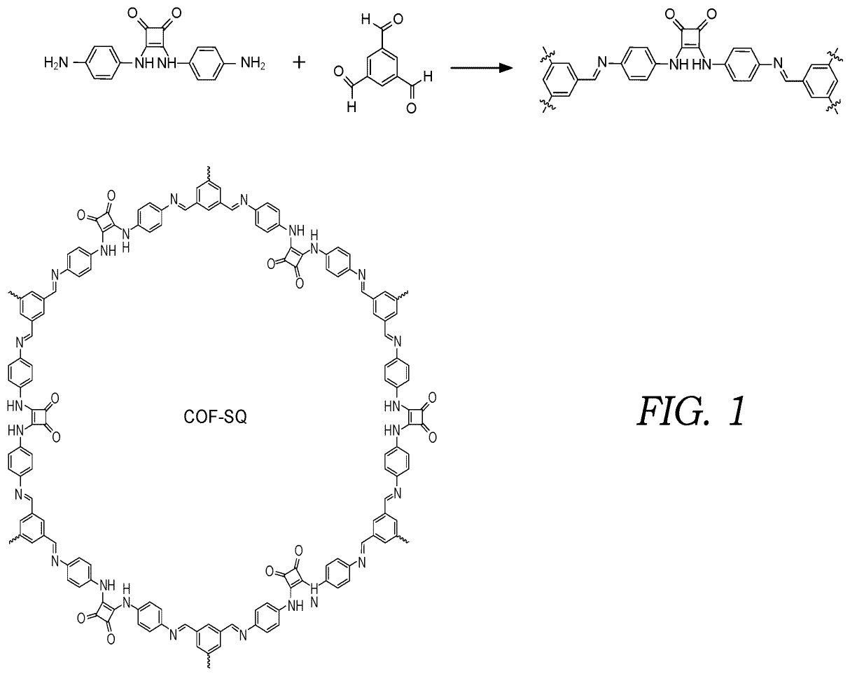

FIG. 1 shows an example of this type of conventional reaction pathway, for forming the covalent organic framework referred to as COF-SQ. In this pathway, for every squaramide functionality introduced into the ring framework of a COF, two additional imines are also introduced, on either side of the squaramide functional group itself.

While the synthesis scheme in FIG. 1 is effective for making COFs that contain a squaramide functionality, such a scheme requires a reagent where the nitrogens from the squaramide functional group are present in the interior of a reagent as secondary amines, while a second set of primary amines are present to participate in the reaction for forming the COF. As a result, the total number of nitrogen atoms in the minimum ring framework path in FIG. 1 is increased, because both the nitrogen atoms from the squaramide functional group and the nitrogen atoms from the imine linkage created during COF formation are present in the framework structure of the COF material. As a practical matter, given that two terminal primary amines are required to be present on either side of the squaramide functional group, only one squaramide functional group will be present in any small molecule reagent that is used to form a COF. Two squaramide functional groups could conceivably be coupled together through a small linker, with subsequent extension of the reagent to either side of this squaramide dimer, but this results in added molecular flexibility and potential electronic communication between the two adjacent squaramide functional groups, with potentially negative implications for the COF framework rigidity, pore/channel size, and sorption behavior. Thus, the constraint of extending the squaramide functional group in a COF synthesis such as the synthesis in FIG. 1 means that, by definition, 50 mol % or less of the nitrogens in the minimum ring framework path of the COF (which correlates with pore and channel size) will correspond to the nitrogens of the squaramide functional group.

In various aspects, synthesis methods are provided for increasing the percentage of nitrogens in the minimum ring framework path of a COF that are nitrogens in a squaramide functional group. To achieve this, the squaramide functional group is not used within a larger reagent molecule. Instead, in various aspects, the squaramide functional group is formed in-situ by the reaction that forms the COF. This is achieved by using a squaric acid alkyl ester as a reagent, such as squaric acid dimethyl ether. Squaric acid dimethyl ether corresponds to squaric acid where both hydroxyl groups are replaced with a methyl ether group. More generally, the hydroxyl groups could be replaced with a methyl ether group, an ethyl ether group, or another small (C₁-C₄)alkyl ether. Due in part to the constrained nature of the squaric acid structure, these alkyl ether groups can react with terminal amines to form COF structures. However, instead of forming the imine groups that are generated when reacting a primary amine with an aldehyde, the reaction of a primary amine with the alkyl ether (such as methyl ether) groups in a squaric acid alkyl ether results in formation of secondary amines. This is facilitated by the presence of an acid catalyst such as p-toluenesulfonic acid, which protonates the alkyl ether oxygen and renders it a good leaving group for nucleophilic displacement by the primary amine. It is noted that if a weak acid like acetic acid is used instead, the desired COF material is not formed. The larger-scale topological aspects of the COF-forming reaction (condensation of the primary amine and the squaric acid alkyl ether) are also facilitated by the presence of added methanol, which (since it has the same structure as the byproduct released during the condensation) assists in maintaining sufficient reversibility of the condensation reaction for good framework regiochemistry and crystallinity to develop. More generally, the alcohol solvent used for the reaction environment can correspond to the same alkoxy chain as the type(s) of ether in the squaric acid alkyl ether. For example, if the squaric acid alkyl ether includes ethoxy groups, then ethanol can be used as the solvent. The amount of acid catalyst present serves to influence the kinetics of the reaction and, as such, also provides a tool for adjusting conditions to obtain good COF product properties, such as crystallinity. As a result, the synthesis method allows for formation of COF compounds where the nitrogens in the squaramide functionality are secondary amines while also increasing the percentage of nitrogens in the minimum ring framework path of the COF that correspond to nitrogens from squaramide functional groups. Depending on the aspect, this can allow for formation of COFs where 51 mol % or more of the nitrogens in the minimum ring framework path are nitrogens that are part of a squaramide functional group, or 60 mol % or more, or 65 mol % or more, or 75 mol % or more, such as up to substantially all of the nitrogens in the minimum ring framework path being nitrogens that are part of a squaramide functional group (˜100 mol %). Thus, the synthesis methods provided herein allow for formation of squaramide-containing COFs that cannot be formed by the conventional synthesis method. In some aspects, the COFs formed according to the synthesis methods provided herein can be substantially free of imines.

### Definitions

The terms “substituent”, “radical”, “group”, and “moiety” may be used interchangeably.

In this discussion, the minimum ring framework path of a covalent organic framework is defined as the minimum number of atoms that must be passed through to traverse around the pore defined by the COF structure and return to the starting atom. As an example, in the COF-SQ structure shown in FIG. 1, the COF framework defines a hexagonal pore as drawn. Each side of the hexagonal pore contains the same number of atoms on each wall of the hexagonal pore. In the COF-SQ structure shown in FIG. 1, the minimum number of atoms that are passed through to travel from one vertex of the hexagon to the next vertex is 19, corresponding to four nitrogen atoms and 15 carbon atoms. Thus, the minimum ring framework path for COF-SQ is 6*19=114 atoms. It is understood that hydrogen atoms are never part of the minimum ring framework path, as hydrogen atoms are always terminal atoms. It is further understood that for ring structures that participate in the minimum ring framework path, only one side of the ring structure needs to be traversed. Thus, the presence of a benzene ring in the minimum ring framework path contributes less than 6 atoms to the number of atoms in the minimum ring framework path. For example, if a benzene ring participates in the COF ring via bonds in the para position, the benzene ring will contribute 4 atoms to the number of atoms in the minimum ring framework path. If a benzene ring participates in the COF ring via bonds in the meta position, the benzene ring will contribute 3 atoms to the number of atoms in the minimum ring framework path.

Because all of the nitrogens in the COF-SQ structure in FIG. 1 correspond to atoms that are in the minimum ring framework path, for the COF-SQ structure in FIG. 1, only 50 mol % of the nitrogens in the COF-SQ structure correspond to nitrogens that are part of a squaramide functionality. The other 50 mol % of the nitrogens correspond to the nitrogens in the imine linkages in FIG. 1.

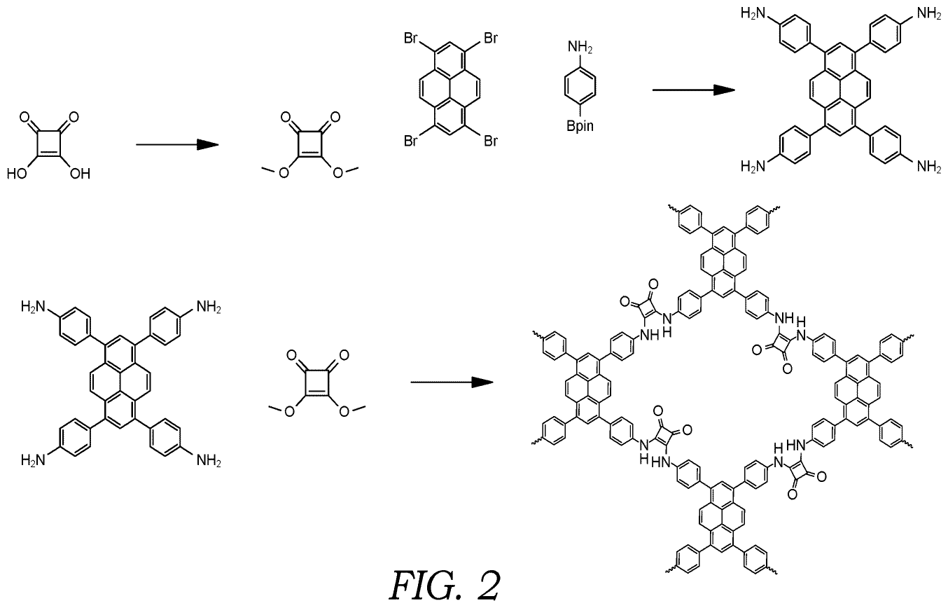

The COF shown in FIG. 2 provides another example of a minimum ring framework path. For the COF shown in FIG. 2, the COF defines a roughly diamond shaped pore, but all four sides of the diamond do not contribute the same number of atoms to the minimum framework path. This is due to the different orientation of the fused ring structure in different sides of the roughly diamond shape. In FIG. 2, two of the sides include 15 atoms in the minimum ring framework path, while the other two sides include 18 atoms. Thus, the minimum ring framework path for the COF in FIG. 2 contains a total 2*15+2*18=66 atoms. In the COF shown in FIG. 2, 100 mol % of the nitrogen atoms in the minimum framework path are nitrogens in a squaramide functional group.

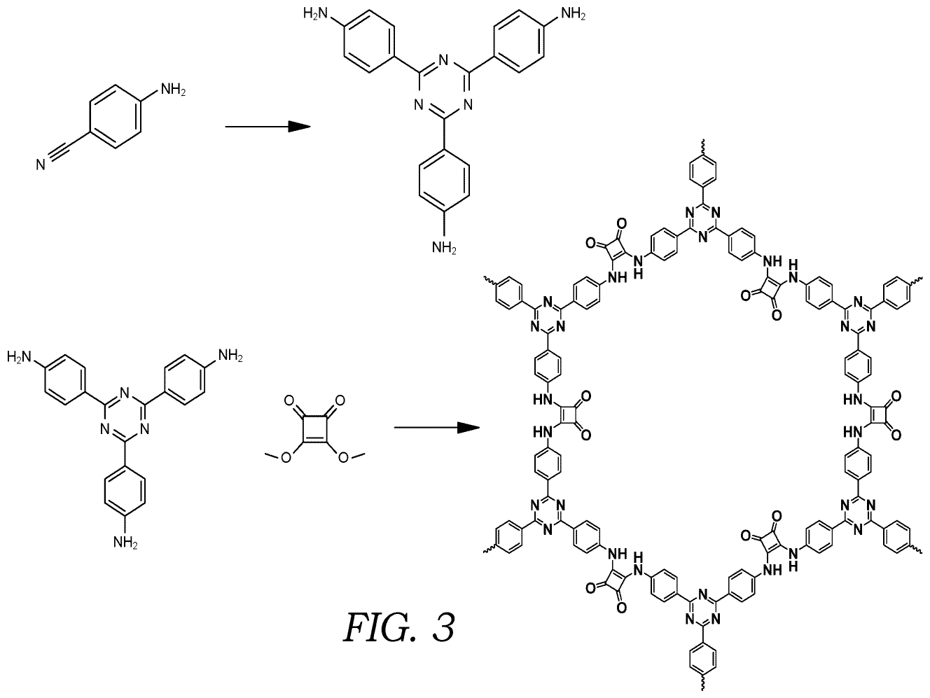

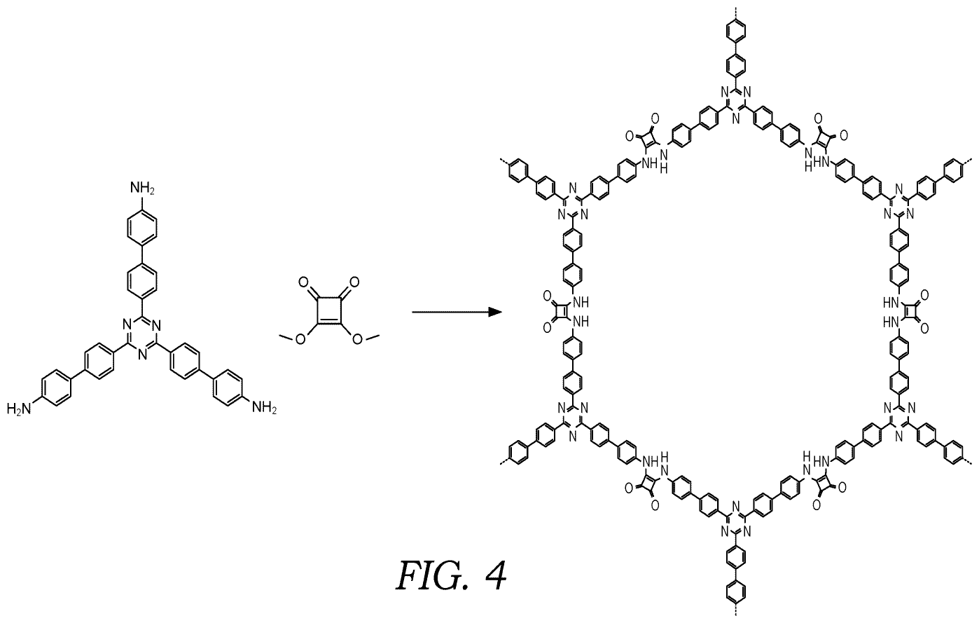

The COF shown in FIG. 3 provides still another example of a minimum ring framework path. For the COF shown in FIG. 3, each side of the hexagonal structure includes 15 atoms in the minimum ring framework path, for a total of 6*15=90 atoms in the minimum ring framework path. It is noted that for the COF in FIG. 3, for the 6-membered rings that contain 3 nitrogens, only one of the nitrogens is part of the minimum ring framework path for a given ring. Thus, 67 mol % of the nitrogens in the minimum ring framework path correspond to nitrogens that are part of a squaramide functional group. It is also noted that for FIG. 2, FIG. 3, and FIG. 4, no specific position of the individual squaramides relative to the COF pore (carbonyl groups versus amine nitrogens pointing inward) is implied by the drawing, which simply shows atomic connectivity.

The COF shown in FIG. 4 provides still another example of a minimum ring framework path. For the COF shown in FIG. 4, each side of the hexagonal structure includes 23 atoms in the minimum ring framework path, for a total of 6*23=138 atoms in the minimum ring framework path. Similar to FIG. 3, for the COF in FIG. 4, 67 mol % of the nitrogens in the minimum ring framework path correspond to nitrogens that are part of a squaramide functional group.

As used herein, and unless otherwise specified, the term “Cₙ” means hydrocarbon(s) having n carbon atom(s) per molecule, wherein n is a positive integer.

As used herein, and unless otherwise specified, the term “hydrocarbon” means a class of compounds containing hydrogen bound to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different values of n.

As used herein, and unless otherwise specified, the term “alkyl” refers to a saturated hydrocarbon radical having from 1 to 14 carbon atoms (i.e. C₁-C₁₄ alkyl), or from 1 to 12 carbon atoms (i.e. C₁-C₁₂ alkyl), or from 1 to 8 carbon atoms (i.e. C₁-C₈alkyl). Examples of alkyl groups include, but are not limited to, methyl, ethyl, propyl, butyl, pentyl, hexyl, heptyl, octyl, decyl, and so forth. The alkyl group may be linear, branched, or cyclic. “Alkyl” is intended to embrace all structural isomeric forms of an alkyl group. For example, as used herein, propyl encompasses both n-propyl and isopropyl; butyl encompasses n-butyl, sec-butyl, isobutyl and tert-butyl and so forth. As used herein, “Cₙ alkyl” refers to methyl (—CH₃), “C₂ alkyl” refers to ethyl (—CH₂CH₃), “C₃ alkyl” refers to propyl (—CH₂CH₂CH₃) and “C₄ alkyl” refers to butyl (e.g. —CH₂CH₂CH₂CH₃, —(CH₃)CHCH₂CH₃, —CH₂CH(CH₃)₂, etc.). As used herein, and unless otherwise specified, the term “alkylene” refers to a divalent alkyl moiety containing 1 to 12 carbon atoms (i.e. C₁-C₁₂ alkylene) in length and meaning the alkylene moiety is attached to the rest of the molecule at both ends of the alkyl unit. For example, alkylenes include, but are not limited to, —CH₂—, —CH₂CH₂—, —CH(CH₃) CH₂—, —CH₂CH₂CH₂—, etc. The alkylene group may be linear or branched.

### Synthesis of Covalent Organic Frameworks Using Squaric Acid Ethers

In various aspects, covalent organic frameworks are formed based on reaction of squaric acid ethers with at least one multi-ring aromatic having three or four primary amine substituents that are sufficiently separated. Sufficiently separated means that the reagent is sufficiently large to prevent the situation where two primary amines in the same reagent molecule react with both ethers of a single squaric acid ester molecule. The reaction results in a covalent organic framework that incorporates squaramide functionality while retaining the amine character of the nitrogens in the squaramide functionality.

The squaric acid ether corresponds to a di-ether with a C₁-C₄ ether attached in place of both hydroxyl groups of squaric acid. Typically the two alkyl ethers will be the same, but this is not required. The squaric acid ether can be made by any convenient method. As an example, to make squaric acid methyl ether, squaric acid can be mixed with methanol, trimethylorthoformate, and trifluoracetic acid. After heating at a heating temperature for a sufficient period of time, the squaric acid methyl ether can be recovered. Various heating temperatures can be used, such as 80° C., or more generally a temperature from 60° C. to 100° C. Similar methods can be used to form other squaric acid ethers.

In order to provide a sufficient distance between primary amines in a reagent molecule, while also providing sufficient rigidity to form a COF structure, the reagent molecule can include a plurality of amine-containing branches that contain a primary amine. Each branch of the reagent molecule that includes a primary amine also includes at least one aromatic ring. Thus, the reagent molecule includes a plurality of aromatic rings that is equal to or greater than the number of primary amines in the reagent molecule.

In addition to the plurality of aromatic rings, the reagent molecule has a central core. The central core can correspond to one or more additional aromatic rings, fused aliphatic rings, branch point central carbons/nitrogens, or a combination thereof. A branch point central carbon is a tertiary or quaternary carbon that is a) not part of a ring structure, and b) that is part of the required pathway to traverse from at least one ring to at least a second ring in the reagent molecule. Examples of branch point central carbons in the amine-containing reagent are the central carbons in the tetraamine reagent 4,4′,4″,4′″-methane-tetrayl-tetraaniline or the triamine reagent 4,4′4″-(ethane-1,1,1-triyl)trianiline. As indicated by the triamine reagent 4,4′4″-(ethane-1,1,1-triyl)trianiline, one of the branches from the quaternary carbon can be a branch that does not include an amine or a ring structure. A branch point central nitrogen is a tertiary nitrogen that is a) not part of a ring structure, and b) that is part of the required pathway to traverse from at least one ring to at least a second ring in the reagent molecule. An example of a branch point central nitrogen is the central nitrogen in triphenylamine, as in the reagent molecule N¹,N¹-bis(4-aminophenyl)benzene-1,4-diamine. An example of a central core corresponding to fused aliphatic rings is a central core of adamantane, as in the reagent 4,4′,4″,4′″-(adamantane-1,3,5,7-tetrayl)tetraaniline.

The minimum number of rings (or rings plus branch point central carbons/nitrogens) depends in part on the number of primary amines in the reagent molecule. For use as a reagent to form a covalent organic framework as described herein, the reagent includes at least one aromatic ring per primary amine, plus a central core that corresponds to at least one additional aromatic ring, fused aliphatic rings, branch point central carbon(s)/nitrogen(s), or a combination thereof. Thus, for a reagent containing three primary amines, the reagent will either include at least four aromatic rings (including one or more aromatic rings in the central core), or at least three aromatic rings and a central core that contains fused aliphatic rings, a branch point central carbon, and/or a branch point central nitrogen. For a reagent containing four primary amines, the reagent will include at least five rings, or at least four rings and a central core that contains at least one of fused aliphatic rings or a branch point central carbon.

In various aspects, the amine-containing reagent has a structure corresponding to a core of one or more central rings, or a branch point central carbon/nitrogen, with either three or four ring-containing branches that each include a primary amine. Examples of structures that can serve as the one or more central rings in the core of the amine-containing reagent include, but are not limited to, benzene (can also be referred to as phenyl), biphenyl (aliphatic bond between two benzene/phenyl rings), pyrene (4 ring fused ring system), biphenylene (two benzene/phenyl rings with two bonds between the ring structures), triphenylene, trinaphthalene, tetranaphthalene, triazine, and adamantane (a multi-cyclic aliphatic ring structure composed of four fused cyclohexane rings). It is noted that adamantane is not an aromatic ring structure, and instead is aliphatic. Fused aliphatic ring structures, such as adamantane, can provide sufficient rigidity for formation of COF structures.

For the branches of the amine-containing reagent, each branch contains at least one ring structure and a primary amine. The primary amine can be attached directly to a ring structure in the branch. In some aspects, the branches for an amine-containing reagent can be characterized based on the number of “heavy” atoms in the branch. In this definition, a “heavy” atom is any atom other than hydrogen. Thus, carbon and nitrogen are heavy atoms under this definition. In such aspects, 50% or more of the heavy atoms in the branches of an amine-containing reagent can correspond to atoms that are part of a ring structure, or 60% or more, such as up to 98%. It is understood that the primary amine in the branch cannot be part of a ring structure, so the percentage of heavy atoms in the branches of the amine-containing reagent will be less than 100%. The number of heavy atoms in each branch of an amine-containing reagent can range from 7 to 30.

Optionally, the one or more central rings or the branch point central carbon in the core of an amine-containing reagent can have one or more alkyl, aryl, or non-reacting (for purposes of the squaramide-forming condensation) heteroatom-containing side chains or substituents in addition to the branches. These substituents, which by definition do not contain a primary amine, are counted as part of the core when determining the number of heavy atoms. In some aspects, 25% or less of the heavy atoms in the core of the amine-containing reagent are heavy atoms in alkyl side chains that do not contain a primary amine. In various aspects, the core of an amine-containing reagent can contain 6 to 26 heavy atoms. Also, optionally, the primary amine-bearing branches can have alkyl, aryl, or non-reacting heteroatom-containing sidechains or substituents. These groups may serve to tune COF properties for sorption applications; for example to decrease pore size or imbue additional polarity.

Optionally, but preferably, each branch of an amine-containing reagent can be the same. It is noted that in this type of optional aspect, two or more different types of amine-containing reagents can be used, such as having a first amine-containing reagent with a first set of branches (with each of the branches in the first set of branches being the same), and a second amine-containing reagent with a second set of branches that are different from the first set of branches (with each of the branches in the second set of branches being the same).

In some aspects, the reagent can include additional nitrogen atoms that do not correspond to the primary amine. These additional nitrogen atoms can be nitrogen atoms that are part of the minimum framework path, nitrogen atoms that are not part of the minimum framework path, or a combination thereof. For example, triazine can be used as a ring in the core of an amine-containing reagent, with branches containing a primary amine and at least one aromatic ring being bonded to the carbon positions of the triazine. Pyridine is another example of a ring structure that can be used as a core and/or as part of a core. It is noted that additional nitrogen atoms different from the primary amines can also be included in the branches. However, in aspects where the final COF structure has a structure where 51 mol % or more of the amines in the minimum ring framework path are the amines from the squaramide functionality, the number of nitrogens in the amine-containing reagent are correspondingly limited. Other heterocyclic atoms that do not participate in the COF framework condensation reaction, such as oxygen or sulfur atoms, may be present in the reagents, cores, or branches.

In some aspects, all of the ring structures in the amine-containing reagent (the component having the three or more primary amines) can be aromatic rings. In other aspects, one or more of the rings in the reagent can be aliphatic (non-aromatic) ring structures.

The squaric acid alkyl ether and the reagent containing the primary amines can be reacted under effective conditions for forming a covalent organic framework material. In the synthesis mixture, the molar ratio of the squaric acid alkyl ether and the reagent containing the primary amines can roughly correspond to the molar ratio of primary amines in the reagent to ethers in the squaric acid alkyl ether. As an example, a squaric acid alkyl ether will have two ether linkages. If the amine-containing reagent has three primary amines, the molar ratio of squaric acid alkyl ether to the amine-containing reagent can be roughly 1.5:1 (3:2). If the amine-containing reagent has four primary amines, the molar ratio of squaric acid alkyl ether to amine-containing reagent can be roughly 2:1. Optionally, either the squaric acid alkyl ether or the amine-containing reagent can be present in excess of the stoichiometric amount, so more generally, the molar ratio of squaric acid alkyl ether to amine-containing reagent can be from 1:1 to 4:1, or 1:1 to 3:1, or 1.2:1 to 4:1, or 1.2:1 to 3:1.

During synthesis of a COF, the squaric acid alkyl ether and the amine-containing reagent are mixed together in the presence of one or more suitable solvents under acidic conditions. The solvents can be any convenient solvents that are suitable for substantially complete solvation of the amine-containing reagent and the squaric acid alkyl ether. Alcohols, aromatic solvents, and mixtures thereof are typically suitable solvents. Examples of suitable solvents are ethanol, tetrahydrofuran, and mixtures thereof. The amount of solvent can be any convenient amount that is sufficient to solvate and/or suspend the squaric acid alkyl ether, the amine-containing reagent, and the acid for the acidic environment, so that the reagents can be well-mixed during the reaction time period. With regard to the acidic environment, a strong organic acid can be added in a molar amount that is comparable to, less than, or greater than the molar amount of the squaric acid alkyl ether. In various aspects, the molar ratio of the strong organic acid to squaric acid alkyl ether can be 0.1 to 12.0 (i.e., from 0.1 to 1 to 12.0 to 1), or 0.1 to 9.0, or 0.1 to 5.0, or 0.5 to 12.0, or 0.5 to 9.0, 0.5 to 5.0, or 0.9 to 12.0, or 0.9 to 9.0, or 0.9 to 5.0, or 1.0 to 5.0, or 0.5 to 3.5, or 0.9 to 3.5, or 1.0 to 3.5. Toluene sulfonic acid (such as o-toluene sulfonic acid or p-toluene sulfonic acid) is an example of a strong organic acid.

The reaction to form the COF can be performed at any convenient pressure. Reaction temperatures of 90° C. to 120° C. can be suitable. The reaction time can range from 1 day to 10 days. It is noted that reaction times of less than a day may allow for forming at least some COF, but reaction times of a day or longer assist with allowing the reaction to substantially reach completion, and also to improve crystallinity. The COF product is purified by extraction with clean solvents and/or supercritical carbon dioxide, the latter of which also functions as a drying agent. The purified materials are typically subjected to final drying steps at elevated temperature under vacuum or inert gas flow, either in a dedicated drying oven or as an in-situ pre-treatment cycle after being loaded into a sorption testing apparatus. In one favored embodiment, the sample is subjected to extraction with clean solvents and then subjected to supercritical carbon dioxide drying immediately before use as a sorbent, but is not heated under vacuum or inert gas flow as a final drying step.

The resulting COF can have a structure that is related to the type of amine-containing reagent that is used. For an amine-containing reagent that includes three primary amines in a planar structure, the resulting COF structure can have two-dimensional layers with roughly hexagonal rings or pores. For an amine-containing reagent that includes four primary amines in a planar structure, the resulting COF structure can have layers with roughly diamond-shaped (or roughly square-shaped) rings or pores. These layered structures are based on forming a COF structure that contains alternating repeat units based on the squaric acid alkyl ether and the amine-containing reagent, respectively. Depending on the nature of the COF, the layers of the COF can have aligned rings or pores, or the rings or pores in different layers can be offset. Amine-containing reagents that include primary amines in a structure that is not planar can form three-dimensional, fused multi-ring structures with the appropriate number of sides per ring (for example, six sides for an amine reagent with three primary amines). The frameworks of these three-dimensional structures may interpenetrate.

The number of heavy atoms in the minimum ring framework path for the COF structure can also be characterized. For COF structures formed from amine-containing reagents that include four primary amines in the para position relative to the amine-bearing aromatic ring's connectivity to the central branch point carbon or central ring(s), the minimum COF ring framework path for the rings or pores in the COF can contain from 52 to 170 heavy atoms, or 52 to 140, or 52 to 114, or 56 to 170, or 56 to 140, or 56 to 114. For COF structures formed from amine-containing reagents that include three primary amines in the para position, the minimum COF ring framework path for the rings or pores in the COF can contain 78 to 210 heavy atoms, or 78 to 180, or 78 to 156, or 90 to 210, or 90 to 180, or 90 to 156, or 102 to 210, or 102 to 180, or 102 to 156. Alternatively, the primary amine-containing reagents may feature primary amines in the meta position relative to the amine-bearing aromatic ring's connectivity to the central branch point carbon or central ring(s). For COF structures formed from amine-containing reagents that include four primary amines in the meta position, the minimum COF ring framework path for the rings of pores in the COF can contain as few as 44 heavy atoms. For COF structures formed from amine-containing reagents that include three primary amines in the meta position, the minimum COF ring framework path can contain as few as 66 heavy atoms. More generally, the minimum COF ring framework path for the rings or pores in the COF can contain 44 to 170 heavy atoms, or 44 to 140, or 44 to 114, or 48 to 170, or 48 to 140, or 48 to 114, or 52 to 170, or 52 to 140, or 52 to 114, or 60 to 170, or 60 to 140, or 60 to 114, or 66 to 210, or 66 to 180, or 66 to 156, or 78 to 210, or 78 to 180, or 78 to 156, or 90 to 210, or 90 to 180, or 90 to 156.

### Applications

One application for the covalent organic frameworks containing squaramide functionality as described herein is for sorption of components from a gas phase flow. As an example, the covalent organic framework containing squaramide functionality can be used for selective adsorption of CO₂ from a gas flow.

A variety of different types of CO₂-containing gases can be used as an input flow for a CO₂ adsorption process. For direct air capture, where air is the input flow gas, a typical CO₂ concentration in air is roughly 400 vppm. More generally, for direct air capture, the CO₂ content of the input flow can be from 300 vppm to 500 vppm. Another typical source of CO₂-containing gas are flue gases from combustion reactions, such as power plants, boilers, furnaces, turbines, or any other type equipment that uses hydrocarbon combustion to generate heat. When the hydrocarbon for the combustion reaction is natural gas, the CO₂ content of the resulting flue gas is typically in the range of 3.0 vol % to 6.0 vol %, or 3.5 vol % to 5.0 vol %. For combustion reactions involving other types of fuels, such as coal, still higher CO₂ contents can be present in the flue gas, such as 8 vol % or higher, or 10 vol % or higher, or 12 vol % or higher, and possibly up to 20 vol % or still higher.

A CO₂-containing gas flow can be used as the input flow for an adsorption process, where the adsorbent is a covalent organic framework containing squaramide functionality, as described herein. During adsorption, the CO₂-containing gas can be exposed to the covalent organic framework in an adsorption environment where the temperature is from 0° C. to 100° C., or 0° C. to 50° C., or 0° C. to 30° C., or 15° C. to 100° C., or 15° C. to 50° C., or 15° C. to 30° C. It is noted that during direct air capture, it is often a goal to perform CO₂ adsorption while modifying the input gas flow as little as possible prior to adsorption, in order to minimize energy costs during the direct air capture process. Thus, in some aspects, the temperature during direct air capture is 0° C. to 50° C., or 0° C. to 30° C., or 15° C. to 50° C., or 15° C. to 30° C.

After sorption of CO₂, the adsorbed CO₂ can be at least partially desorbed to regenerate the adsorbent to allow for a cyclic process. One option for doing this is to increase the temperature in the sorbent environment. For example, desorption can occur by sufficiently increasing the temperature while still exposing the sorbent to the CO₂-containing gas flow, or alternatively, exposing the sorbent to another non-CO₂-containing gas flow, or to vacuum, with or without increasing the temperature. Desorption can be performed at a temperature of 50° C. to 180° C., or 50° C. to 100° C., or 90° C. to 150° C.

### EXAMPLES

An example of a procedure for making a squaric acid methyl ether is described in Claveau et al. Chem Commun. 2018, 54, 3231. An example of a procedure for making 1,3,6,8-tetrakis(4-aminophenyl-pyrene), Py-NH₂ is described in Ascherl et al. J Am Chem Soc, 2019, 141, 15693. An example of a procedure for making 2,4,6-tris(4-aminophenyl)-1,3,5-triazine (TAPT) is described in Zhang et al. Angew. Chem. Int. Ed. Engl. 2018, 57, 16754. An example of a procedure for making 4,4′,4″-(1,3,5-triazine-2,4,6-triyl)tris [(1,1′-biphenyl)-4-amine], TTBA, is described in Dey et al. J. Am. Chem. Soc. 2017, 139, 13083.

### Example 1—Synthesis of NUS-17, Phenyl-Pyrene-Squaramide COF

Squaric acid methyl ether was made by the following method: A 100 mL round-bottomed flask containing a magnetic stirring bar under an argon atmosphere was charged with squaric acid (4.00 g, 35.07 mmol). Dry methanol (MeOH) (40 mL), followed by trimethyl orthoformate (11.5 mL, 105.2 mmol) and trifluoracetic acid (536.8 μL, 7.01 mmol-20 mol %), were then added via syringe. The flask was fitted with a condenser, and the reaction mixture was heated at reflux temperature (80° C.) for 48 hr and then cooled to room temperature. The volatiles were removed in vacuo, and the residue obtained was purified by flash column chromatography (hexanes:ethylacetate, 2:1 v/v) to give the product squaric acid methyl ether, SA-OMe, 3,4-dimethoxy-3-cyclobutene-1,2-dione, as a white solid (yield: 84%). Separately, 1,3,6,8-Tetrabromopyrene (0.74 g, 1.4 mmol, 1.0 eq.), 4-aminophenylboronic acid pinacol ester (1.5 g, 6.9 mmol, 4.8 eq.), K₂CO₃ (1.1 g, 7.9 mmol, 5.5 eq.), and Pd(PPh₃)₄ (Tetrakis(triphenylphosphine) palladium, 165 mg, 0.14 mmol, 10 mol %) were added to a flask under argon atmosphere. Degassed 1,4-dioxane (16 mL) and H₂O (4 mL) were added to dissolve the solid, and the mixture was heated to reflux (388 K) for 3 days. After the mixture cooled to room temperature, a large excess amount of H₂O was added. The resulting light yellowish precipitate was collected via filtration and washed with H₂O and methanol. The crude product was purified by dissolving in hot 1,4-dioxane and precipitating in water. The precipitate was filtered and dried under high vacuum to afford 1,3,6,8-tetrakis(4-aminophenyl-pyrene), Py-NH₂, a bright yellow powder (yield: 81%).

The Py-NH₂ and SA-OMe were then used to make the COF that is labeled as NUS-17 in FIG. 2. FIG. 2 generally shows the synthesis scheme. To make NUS-17, Py-NH₂ (22.35 mg, 0.04 mmol) and SA-OMe (11 mg, 0.08 mmol) were placed in a Pyrex tube (10 mL). Then 2 mL MeOH was added to the tube and the mixture was sonicated for 10 min. After that, p-toluenesulfonic acid (PTSA) (45 mg, 0.23 mmol) was added to the suspended solids and the mixture was sonicated for another 10 min. The tube was flame-sealed after three freeze-pump-thaw cycles at 77 K and the mixture was allowed to react for 3 days at 90° C. The obtained yellow solids were washed with dimethylformamide (DMF) and tetrahydrofuran (THF) three times and then immersed in ethanol. The ethanol-wetted sample was subjected to 4 cycles in a supercritical CO₂ dryer (5° C./6 mPa) followed by a final activation/drying step of (35° C./>7.5 mPa). The resulting material was NUS-17.

### Example 2—Synthesis of NUS-18, Phenyl-Triazine-Squaramide COF

2,4,6-tris(4-aminophenyl)-1,3,5-triazine (TAPT) was made by the following method. 4-aminobenzonitrile (772.0 mg, 6.5 mmol) was added into a 50.0 mL Schlenk flask, which was evacuated then backfilled with argon. CHCl₃ (10 mL) was then added followed by CF₃SO₃H (2.0 mL, 22.2 mmol, dropwise for 20 min) in an ice-water bath. The reaction was stirred for 24 hr at room temperature (25° C.). Afterwards, 20.0 mL of distilled water was added, and the reaction mixture was neutralized by 2 M NaOH solution. A pale-yellow product, 2,4,6-tris(4-aminophenyl)-1,3,5-triazine (TAPT) was washed with deionized water several times and dried in an 80° C. vacuum oven (yield: 92%).

The TAPT and SA-OMe were then used to make the COF that is labeled as NUS-18 in FIG. 3. FIG. 3 generally shows the synthesis scheme. To make NUS-18, TAPT (28.35 mg, 0.08 mmol) and SA-OMe (Example 1, 18 mg, 0.12 mmol) were placed in the Pyrex tube (10 mL). Then the 0.2 mL MeOH and 1.8 mL o-dichlorobenzene (oDCB) were added to the tube and sonicated for 10 min. After that, PTSA (25 mg, 0.12 mmol) was added to the suspended solids and sonicated for another 10 min. Then the tube was flame-sealed after three freeze-pump-thaw cycles at 77 K and allowed for a reaction at 120° C. for 3 days. The obtained dark red solids were washed with THE three times and then immersed in ethanol for further supercritical CO₂ activation as described in Example 1. The resulting material was NUS-18.

### Example 3—Synthesis of NUS-19, Biphenyl-Triazine-Squaramide COF

4,4′,4″-(1,3,5-triazine-2,4,6-triyl)tris [(1,1′-biphenyl)-4-amine], TTBA, was made by the following method. 0.5 g (8.58 mmol) 4-aminobenzonitrile was added to a Schlenk tube at 0° C. Then 2.3 mL (25.74 mmol) trifluoromethanesulfonic acid was added dropwise for 20 min maintaining the temperature at 0° C. The resultant mixture was heated in an inert atmosphere at 100° C. for 12 h with constant stirring. Then 30 mL of distilled water was added to the mixture, and it was neutralized by adding 2M NaOH solution until pH reached 7. The resultant precipitate, 4,4′,4″-(1,3,5-triazine-2,4,6-triyl)tris [(1,1′-biphenyl)-4-amine], TTBA, was filtered and washed several times with distilled water and dried in 80° C. vacuum oven (yield: 80.46%).

The TTBA and SA-OMe were then used to make the COF that is labeled as NUS-19 in FIG. 4. FIG. 4 generally shows the synthesis scheme. To make NUS-19, TTBA (35 mg, 0.06 mmol) and SA-OMe (12 mg, 0.09 mmol) were placed in a Pyrex tube (10 mL). Then 0.1 mL MeOH and 1.9 mL oDCB were added to the tube and the mixture was sonicated for 10 min. Afterwards, PTSA (15 mg, 0.09 mmol) was added to the suspended solids and they were sonicated for another 10 min. The tube was then flame-sealed after three freeze-pump-thaw cycles at 77 K and the mixture was allowed to react at 120° C. for 3 days. The obtained yellow solids were washed with THE three times and then immersed in ethanol for further supercritical CO₂ treatment for activation as described in Example 1. The resulting material was NUS-19.

### Example 4—Additional Characterization

Fundamental structural properties of the COFs (as prepared and purified by supercritical CO₂ extraction above) are summarized in Table 1. Brunauer-Emmett-Teller (BET) surface area and pore diameter (NLDFT method was used) were determined by N₂ sorption at 77K using a Micromeritics ASAP 2020 instrument equipped with commercial software for data calculation, and analysis. Before each measurement, the samples were degassed at 373K for 24 hours. Pore diameters were also estimated from the N₂ adsorption isotherm using a nonlocal density functional theory fitting method.

TABLE 1
Structural Properties

| | NUS-17 | NUS-18 | NUS-19 |
|---|---|---|---|
| Pore geometry | rhombic | hexagonal | hexagonal |
| Spacer/branch point | phenyl/pyrene | phenyl/triazine | biphenyl/triazine |
| Color | yellow | dark red | orange |
| BET Surface area, m²/g | 411 | 527 | 585 |
| BET Porosity/isotherm | Micro (Type 1) | Meso (Type IV) | Meso (Type IV) |
| Pore diameter (nm, based | 1.1 and 1.3 | 1.2 and 2 | 1.3 and 2.7 |
| on NLDFT method in N₂ | | | |
| adsorption, 77 K) | | | |
| (100) and (001) peak in | 4.57, 23.61 | 3.45, 26.57 | 2.33, 25 |
| PXRD (°) | | | |
| Space Group | P1 | P3 | P3 |
| Structure a, b, c (Å) | 39.35, 39.96, 4.0 | 29.27, 29.27, 3.8 | 44.13, 44.13, 3.5 |
| Structure α, β, γ (°) | 87.31, 101.02, 92.34 | 90, 90, 120 | 90, 90, 120 |
| Stacking | AB (staggered) | AB (staggered) | AB (staggered) |
| Structure agreement factors | Rₚ 3.72%, R_{wp} | Rₚ 4.3%, R_{wp} | R_{wp} 3.68%, Rₚ |
| | 4.71% | 5.45% | 2.63% |

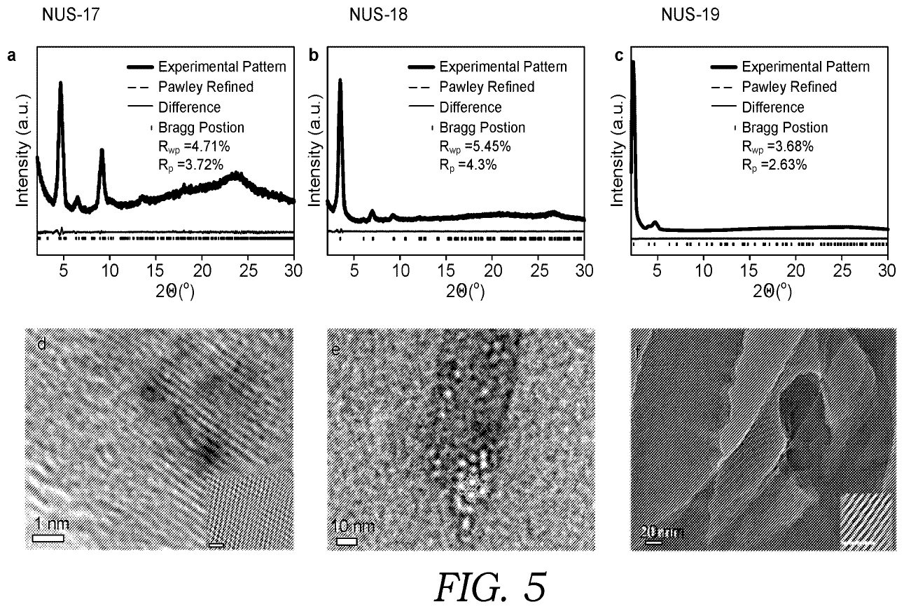

FIG. 5 shows Powder X-ray diffraction (PXRD) patterns of materials prepared by the representative procedures of Example 1 (NUS-17), Example 2 (NUS-18), and Example 3 (NUS-19). Diffraction patterns were collected using a Rigaku Miniflex 600 diffractometer (Cu Kα λ=1.540598 Å) with an operating power of 40 kV, 15 mA, and a scan rate of 2.0° min⁻¹. The data were collected in a two-theta range of 2° to 30°. Pawley structural refinements were performed on the obtained PXRD data using the Reflex Module in Materials Studio software (Andzelm et al. Chem. Phys. Lett. 2001, 335, 321). COF structures were optimized by the Forcite module, then the refinements of the PXRD patterns were conducted in the Reflex module using 2 thetas from 2° to 30°. The integrated intensities were extracted with Pseudo-Voigt profile. Pawley refinement was utilized to optimize the lattice parameters iteratively until the R_{P} and R_{WP} values converged. All three COFs showed staggered (AB stacking) based on pore size distribution and agreement with simulated PXRD patterns.

FIG. 5 also shows corresponding high-resolution transmission electron microscopy (HR-TEM) images. Images were taken following sonication in hexane for 30 mins. Observations were performed on a JEOL JEM2100 microscope operated at 200 kV (Cs 1.0 mm, point resolution 0.23 nm). Images were recorded with a Gatan Orius 833 CCD camera (resolution 2048×2048 pixels, pixel size 7.4 μm) and demonstrate the regular and porous structures of the squaramide-based COFs. Distinct spacing of the lattice fringes were seen for NUS-17 (˜4 Å, (001) plane) and NUS-19 (2.7 nm, (100) plane). Ordered mesopores were observed in the split single-layer NUS-18, and were well-matched with the simulated pore structure.

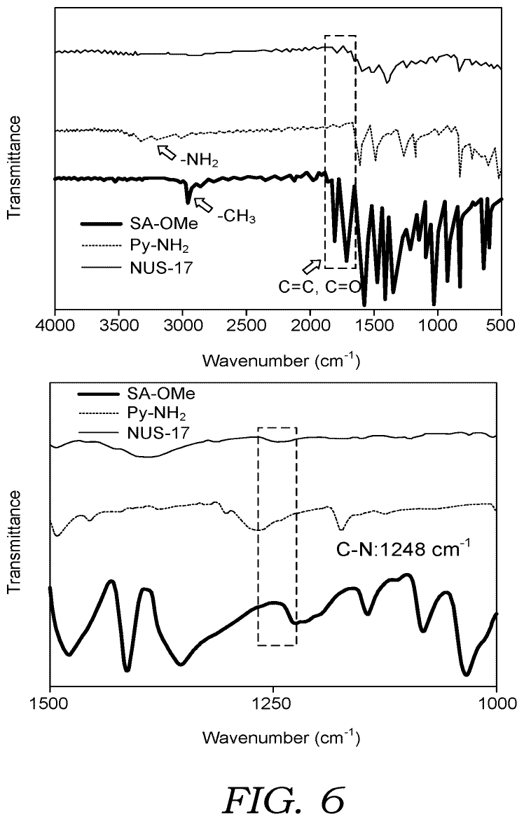

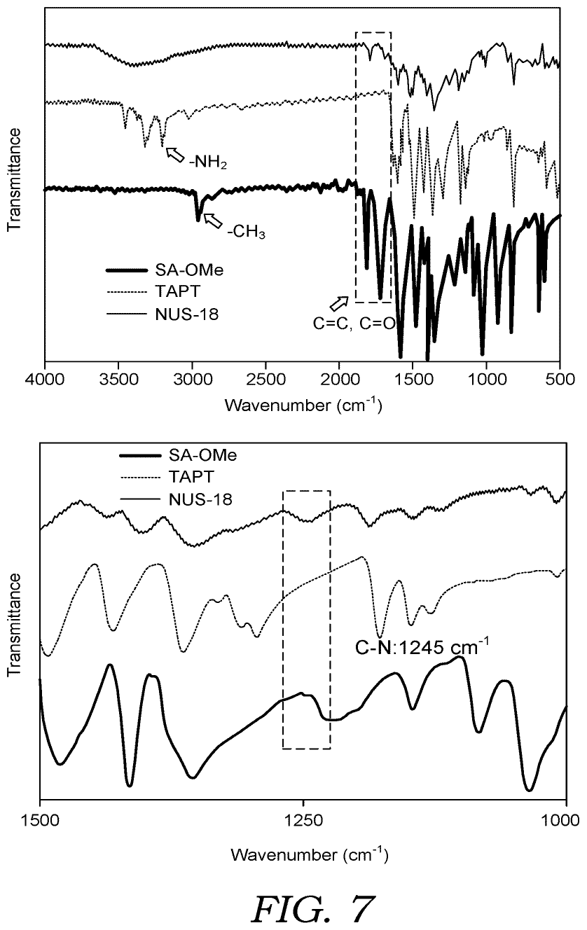

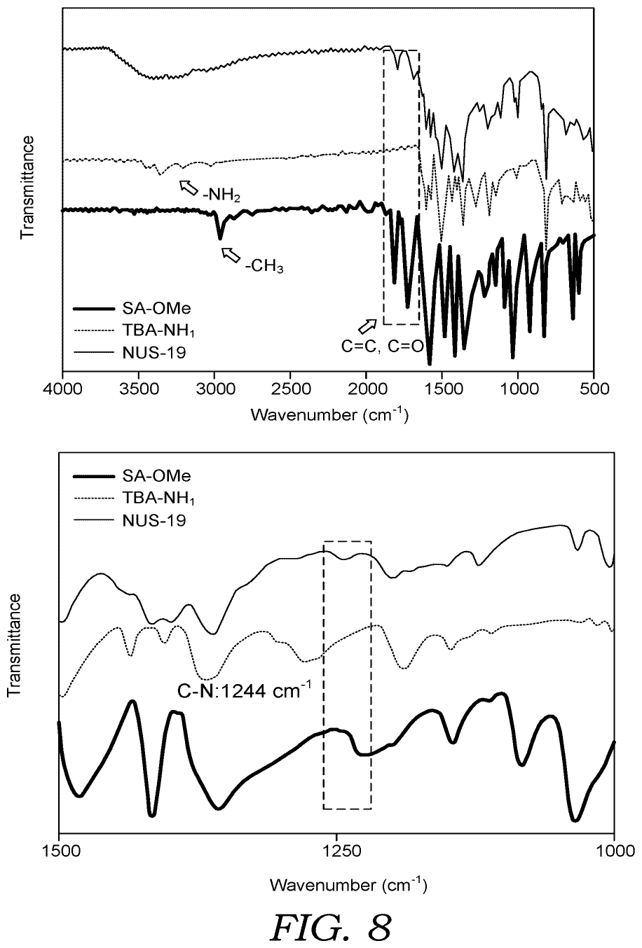

The synthesis of COFs with a squaramide linkage was monitored using Fourier-transform infrared (FT-IR) spectroscopy on materials prepared by the representative procedures of Example 1 (FIG. 6, NUS-17), Example 2 (FIG. 7, NUS-18), and Example 3 (FIG. 8, NUS-19). FTIR was performed with a Bio-Rad FTS-3500 ARX FTIR spectrometer. The disappearance of vibrational absorbance bands at 2967 cm⁻¹ (methyl vc-H stretch) in SA-OMe, along with the absence of characteristic peaks of amino at 3322 cm⁻¹ and 3214 cm⁻¹, was observed in the spectra after crystallization.

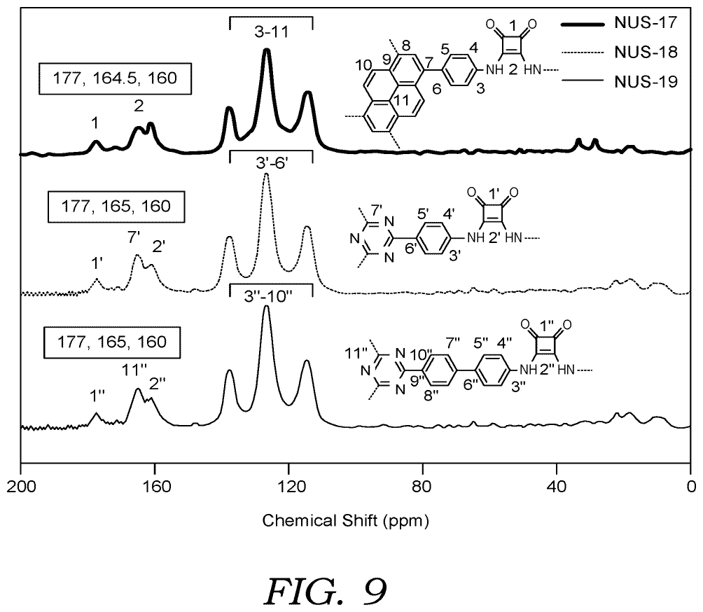

Solid-state ¹³C NMR (ssNMR) experiments were also conducted on a 14.1 T Bruker Advance III HD 600 MHz spectrometer utilizing a 1.9 mm Bruker HXY probe at a MAS frequency of 30 kHz. (FIG. 9). The squaramide carbonyl and olefin appear at ˜177 and ˜160 ppm, respectively. The AB stacking of NUS-17 is indicated by the split ssNMR olefin peak (sharp peak at 160 ppm; broad peak at 164.5 ppm). This cannot be observed cleanly for NUS-18 and -19 due to overlap with the triazine carbon.

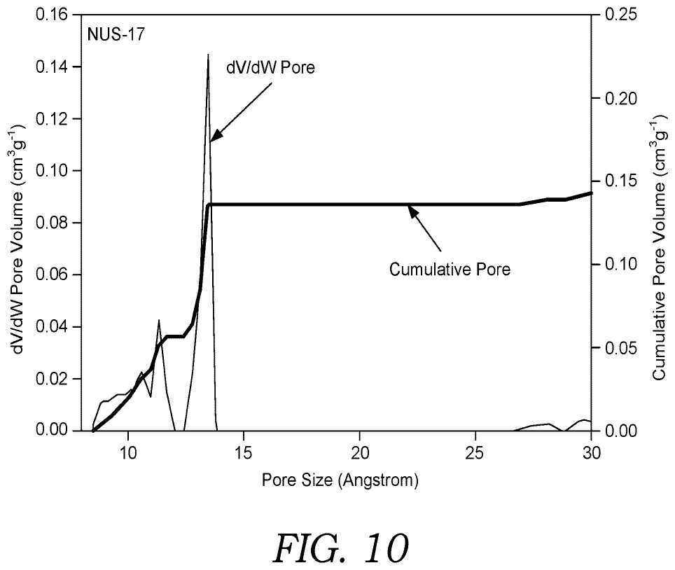

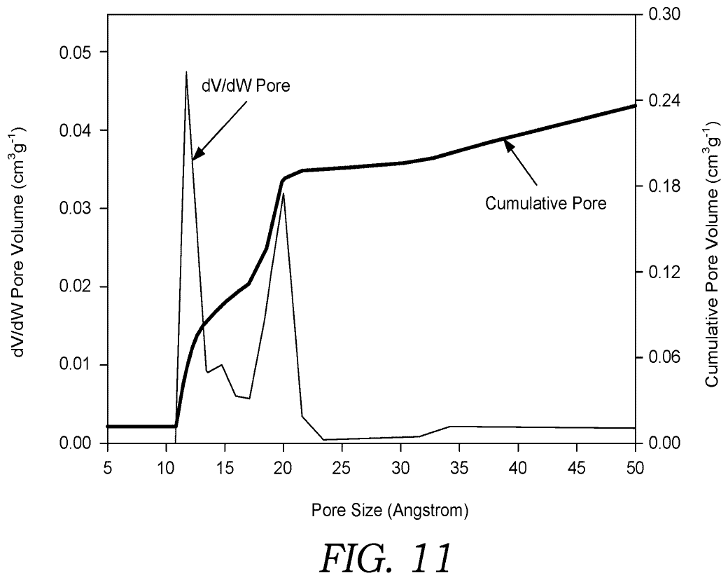

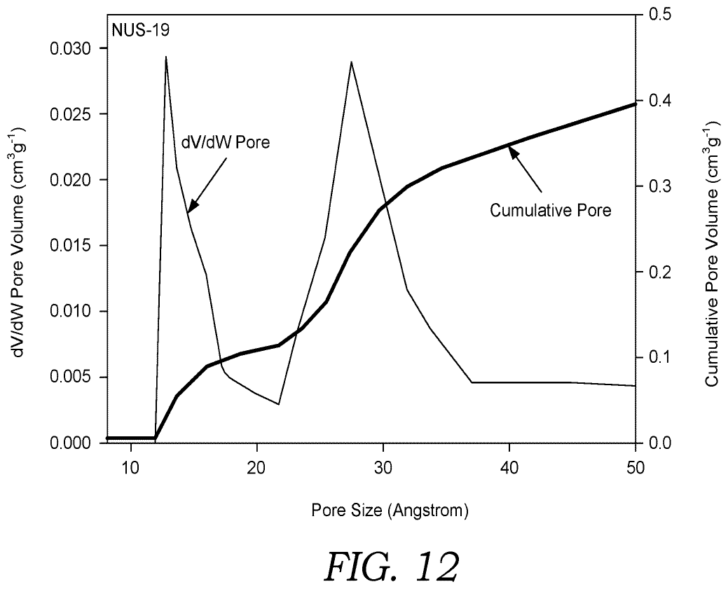

FIG. 10 (NUS-17), FIG. 11 (NUS-18), and FIG. 12 (NUS-19) show N₂ adsorption isotherms at 77K for samples of the synthesized COF materials. In the 77K nitrogen adsorption measurements, NUS-17 showed a type I adsorption isotherm with a rapid increase in N₂ uptakes at P/P₀<0.1 indicating a microporous structure. NUS-18 and NUS-19 exhibited type IV patterns with steep adsorption at P/P₀<0.2 and P/P₀<0.4, followed by a steady increase between P/P₀=0.2 and P/P₀=0.4 to P/P₀=1, respectively. Desorption hysteresis at P/P₀=0.5 in NUS-18 suggested blocked mesopores. BET specific surface areas were calculated and are shown in Table 1 along with pore diameters estimated from the N₂ adsorption isotherm using a nonlocal density functional theory fitting method (11.1 Å and 13 Å in NUS-17, 12 Å and 20 Å in NUS-18, and 13 and 27 Å in NUS-19). These values are in agreement with AB stacked structures (calculated pore diameters for AA stacked NUS-17, NUS-18, and NUS-19 are 2, 2.7, and 4 nm, respectively).

Table 3 shows CO₂ uptake data at 298K for samples of the COF materials made according to Example 1, Example 2, and Example 3. The isotherm dry CO₂ gas sorption experiments (line 2) were performed using a Micromeritics ASAP 2020 instrument equipped with commercial software for data calculation and analysis. The temperatures were controlled by soaking the sample tube in a circulating water bath. Before each measurement, the samples were degassed at 373 K for 24 hr.

Mixed gas breakthrough experiments (Table 3, line 2; 15:85 CO₂/N₂ with varying relative humidity) were conducted using scaled-up batches of COFs prepared and purified identically to Examples 1-3 (surface area was not measured). These samples were stored in a drybox at room temperature under N₂ for 8 months prior to testing and subjected to supercritical CO₂ extraction/purification shortly before breakthrough testing. Data is given in Table 3. The experiment was conducted in an apparatus utilizing a stainless-steel column (4.6 mm inner diameter×100 mm length). Before the breakthrough experiment, the column was packed with the powdered COFs (ground to ˜0.5 μm particle size; 0.2 g NUS-17, 0.28 g NUS-18, and 0.3 g NUS-19), which were activated with a He flow (5 mL min⁻¹) by heating to 373 K at a rate of 20 K min⁻¹ and holding for 12 h. The column was subsequently cooled to room temperature at a rate of 20 K min⁻¹. Feed gas (15% CO₂ in N₂, either dry or humidified, Standard gas from Airliquid) was then passed through a bypass line at room temperature at a rate of 22 mL min⁻¹ for a period of 1 h (22% relative humidity (RH) feed gas was humidified by bubbling through a saturated aqueous potassium acetate solution; 53% RH through saturated aqueous magnesium nitrate; 100% RH through pure water). The balanced gas was then introduced into the sample column and the outlet gas from the column was monitored using a mass spectrometer (Hiden QGA quantitative gas analysis system) with Ar (5 mL min⁻¹) as the interior label gas. Once the outlet gas reached equilibrium (30 minutes), the inlet gas was changed to He (5 mL min⁻¹) immediately for desorption at room temperature. After the experiments, the column was heated up to 373 K for further activation.

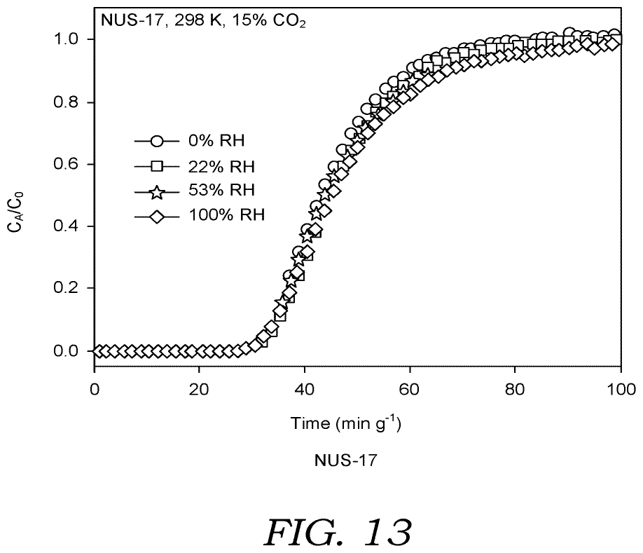

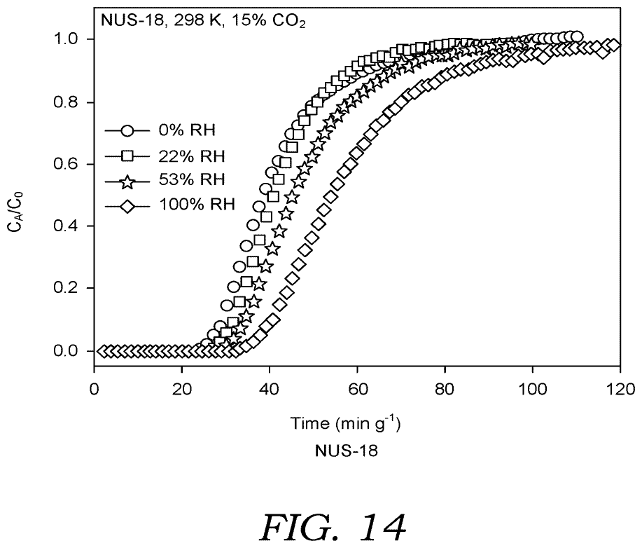

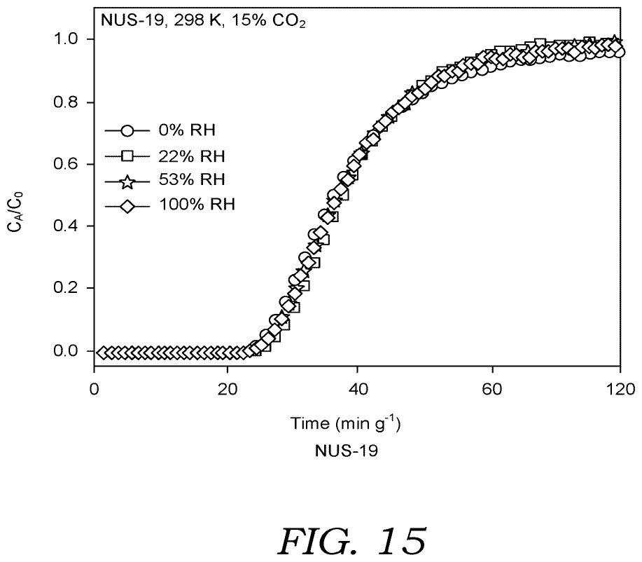

As shown in Table 3 and FIGS. 13 to 15, each of the COF materials had the ability to adsorb CO₂, including CO₂ adsorption in the presence of up to 100% relative humidity (RH). FIGS. 13-15 show mixed gas breakthrough curves for the three COFs performed at 298 K using 15% CO₂ in N₂ feed gas, either dry or pre-humidified to three different relative humidities. C_{A}/C₀ represents the amount of CO₂ detected at the sorbent column exit as a function of inlet concentration as plotted over experiment time (C_{A}/C₀=0 means all entering CO₂ has been adsorbed; C_{A}/C₀=1 means that no entering CO₂ has been adsorbed).

TABLE 3
CO₂ Uptake

| | NUS-17 | NUS-18 | NUS-19 |
|---|---|---|---|
| 298 K dry CO₂ | 0.44 | 0.40 | 0.25 |
| uptake, 0.15 bar, mmol/g | | | |
| 298 K CO₂ | 0.31, 0.35, | 0.30, 0.38, | 0.20, 0.22, |
| uptake from breakthrough | 0.37, 0.39 | 0.42, 0.62 | 0.20, 0.21 |
| sorption, 0.15 bar, | | | |
| at 0, 22, 53, and 100% | | | |
| relative humidity (mmol/g) | | | |

### ADDITIONAL EMBODIMENTS

Embodiment 1. A method for forming a covalent organic framework composition that incorporates squaramide functionality, comprising: reacting a squaric acid alkyl ether with at least one amine-containing reagent in the presence of an organic strong acid under framework synthesis conditions to form a covalent organic framework having squaramide functional groups, wherein nitrogen atoms of the squaramide functional groups in the covalent organic framework composition correspond to 51% or more of nitrogen atoms in the minimum ring framework path of a ring defined by the covalent organic framework, wherein the at least one amine-containing reagent comprises a central core and a plurality of amine-containing branches, the plurality of branches being 3 branches or 4 branches, wherein each branch in the plurality of amine-containing branches comprises a primary amine and at least one aromatic ring, wherein the central core comprises one or more additional aromatics rings, a plurality of fused aliphatic rings, a branch point central carbon, a branch point central nitrogen, or a combination thereof, and wherein the ether groups of the squaric acid alkyl ether each contain 1 to 4 carbon atoms.

Embodiment 2. The method of Embodiment 1, wherein each branch in the plurality of branches of the at least one amine-containing reagent comprises 7 to 30 heavy atoms, each branch in the plurality of branches of the at least one amine-containing reagent optionally being the same.

Embodiment 3. The method of any of the above embodiments, wherein the nitrogen atoms of the squaramide functional groups in the covalent organic framework correspond to 65% or more of the nitrogen atoms in the minimum ring framework path of a ring defined by the covalent organic framework, or wherein the nitrogen atoms of the squaramide functional groups in the covalent organic framework material correspond to 75% or more of the nitrogen atoms in the minimum ring framework path of a ring defined by the covalent organic framework.

Embodiment 4. The method of any of the above embodiments, wherein the minimum ring framework path of a ring defined by the covalent organic framework is substantially free of imines.

Embodiment 5. The method of any of Embodiments 1 to 4, wherein the central core of the at least one amine-containing reagent comprises 6 to 26 heavy atoms, the central core optionally comprising at least one additional aromatic ring.

Embodiment 6. The method of any of Embodiments 1 to 4, wherein the central core of the at least one amine-containing reagent comprises at least one of a branch point central carbon and a branch point central nitrogen, the central core of the at least one amine-containing reagent optionally further comprising one or more alkyl substituents.

Embodiment 7. The method of any of the above embodiment, wherein the minimum ring framework path of a ring defined by the covalent organic framework comprises 44 heavy atoms to 170 heavy atoms, or 52 heavy atoms to 170 heavy atoms, or preferably 60 heavy atoms to 170 heavy atoms; or wherein the minimum ring framework path of a ring defined by the covalent organic framework comprises 66 heavy atoms to 210 heavy atoms, or 78 heavy atoms to 210 heavy atoms, or preferably 90 heavy atoms to 210 heavy atoms.

Embodiment 8. The method of any of the above embodiments, wherein 25% or less of the heavy atoms in the central core of the at least one amine-containing reagent are heavy atoms in alkyl side chains.

Embodiment 9. The method of any of the above embodiments, wherein the central core of the at least one amine-containing reagent is selected from the group consisting of benzene, biphenyl, pyrene, biphenylene, triphenylene, trinaphthalene, triphenylamine, tetranaphthalene, triazine, and adamantane.

Embodiment 10. The method of any of the above embodiments, wherein the strong organic acid is o-toluenesulfonic acid, p-toluenesulfonic acid, or a combination thereof.

Embodiment 11. A covalent organic framework made according to the method of any of Embodiments 1 to 10.

Embodiment 12. A covalent organic framework composition that incorporates squaramide functionality, comprising: a covalent organic framework containing squaramide functional groups, the nitrogen atoms of the squaramide functional groups in the covalent organic framework corresponding to 51% or more of nitrogen atoms in the minimum ring framework path of a ring defined by the covalent organic framework, the minimum ring framework path further incorporating atoms from a plurality of aromatic rings, a number of rings in the plurality of aromatic rings being equal to or greater than a number of nitrogen atoms of squaramide functional groups in the minimum ring framework path, wherein i) the minimum ring framework path of a ring defined by the covalent organic framework comprises 44 heavy atoms to 170 heavy atoms, or 52 heavy atoms to 170 heavy atoms, or preferably 60 heavy atoms to 170 heavy atoms, or ii) the minimum ring framework path of a ring defined by the covalent organic framework comprises 66 heavy atoms to 210 heavy atoms, or 78 heavy atoms to 210 heavy atoms, or preferably 90 heavy atoms to 210 heavy atoms.

Embodiment 13. The covalent organic framework composition of Embodiment 12, wherein the nitrogen atoms of the squaramide functional groups in the covalent organic framework correspond to 65% or more of the nitrogen atoms in the minimum ring framework path of a ring defined by the covalent organic framework, or wherein the nitrogen atoms of the squaramide functional groups in the covalent organic framework correspond to 75% or more of the nitrogen atoms in the minimum ring framework path of a ring defined by the covalent organic framework.

Embodiment 14. The covalent organic framework composition of Embodiment 12 or 13, wherein the minimum ring framework path of a ring defined by the covalent organic framework is substantially free of imines.

Embodiment 15. A method of adsorbing CO₂, comprising exposing a covalent organic framework composition according to any of Embodiments 12 to 14 or a composition made according to any of Embodiments 1 to 10, to a gas phase environment comprising CO₂, the gas phase environment optionally comprising a) 6.0 vol % or less of CO₂, or b) 8.0 vol % to 20 vol % of CO₂.

Additional Embodiment A. The method of any of Embodiments 1 to 10, wherein the primary amines in the branches of the at least one amine-containing reagent are in the para position relative to the connectivity to the central core.

Additional Embodiment B. The method of any of Embodiments 1 to 10, wherein a molar ratio of the squaric acid alkyl ether to the strong organic acid is 0.1 to 12.0.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A method for forming a covalent organic framework composition that incorporates squaramide functionality, comprising:
reacting a squaric acid alkyl ether with at least one amine-containing reagent in the presence of an organic strong acid under framework synthesis conditions to form a covalent organic framework having squaramide functional groups,
wherein nitrogen atoms of the squaramide functional groups in the covalent organic framework correspond to 51% or more of nitrogen atoms in the minimum ring framework path of a ring defined by the covalent organic framework,
wherein the at least one amine-containing reagent comprises a central core and a plurality of amine-containing branches, the plurality of branches being 3 branches or 4 branches,
wherein each branch in the plurality of amine-containing branches comprises a primary amine and at least one aromatic ring,
wherein the central core comprises one or more additional aromatic rings, a plurality of fused aliphatic rings, a branch point central carbon, a branch point central nitrogen, or a combination thereof, and
wherein the ether groups of the squaric acid alkyl ether each contain 1 to 4 carbon atoms.

2. The method of claim 1, wherein each branch in the plurality of branches of the at least one amine-containing reagent is the same.

3. The method of claim 1, wherein each branch in the plurality of branches of the at least one amine-containing reagent comprises 7 to 30 heavy atoms.

4. The method of claim 1, wherein the nitrogen atoms of the squaramide functional groups in the covalent organic framework correspond to 65% or more of the nitrogen atoms in the minimum ring framework path of a ring defined by the covalent organic framework.

5. The method of claim 1, wherein the minimum ring framework path of a ring defined by the covalent organic framework is substantially free of imines.

6. The method of claim 1, wherein the central core of the at least one amine-containing reagent comprises 6 to 26 heavy atoms.

7. The method of claim 1, wherein the central core of the at least one amine-containing reagent comprises at least one of a branch point central carbon and a branch point central nitrogen.

8. The method of claim 7, wherein the central core of the at least one amine-containing reagent further comprises one or more alkyl substituents.

9. The method of claim 1, wherein the central core comprises at least one additional aromatic ring.

10. The method of claim 1, wherein the minimum ring framework path of a ring defined by the covalent organic framework comprises 44 heavy atoms to 170 heavy atoms, or wherein the minimum ring framework path of a ring defined by the covalent organic framework comprises 66 heavy atoms to 210 heavy atoms.

11. The method of claim 1, wherein the minimum ring framework path of a ring defined by the covalent organic framework comprises 52 heavy atoms to 170 heavy atoms, or wherein the minimum ring framework path of a ring defined by the covalent organic framework comprises 78 heavy atoms to 210 heavy atoms.

12. The method of claim 1, wherein 25% or less of the heavy atoms in the central core of the at least one amine-containing reagent are heavy atoms in alkyl side chains.

13. The method of claim 1, wherein the central core of the at least one amine-containing reagent is selected from the group consisting of benzene, biphenyl, pyrene, biphenylene, triphenylene, trinaphthalene, triphenylamine, tetranaphthalene, triazine, and adamantane.

14. The method of claim 1, wherein the strong organic acid is o-toluenesulfonic acid, p-toluenesulfonic acid, or a combination thereof.

15. A covalent organic framework composition that incorporates squaramide functionality, comprising:
a covalent organic framework containing squaramide functional groups, the nitrogen atoms of the squaramide functional groups in the covalent organic framework material corresponding to 51% or more of nitrogen atoms in the minimum ring framework path of a ring defined by the covalent organic framework, the minimum ring framework path further incorporating atoms from a plurality of aromatic rings, a number of rings in the plurality of aromatic rings being equal to or greater than a number of nitrogen atoms of squaramide functional groups in the minimum ring framework path,
wherein i) the minimum ring framework path of a ring defined by the covalent organic framework comprises 44 heavy atoms to 170 heavy atoms, or ii) the minimum ring framework path of a ring defined by the covalent organic framework comprises 66 heavy atoms to 210 heavy atoms.

16. The covalent organic framework composition of claim 15, wherein the nitrogen atoms of the squaramide functional groups in the covalent organic framework correspond to 65% or more of the nitrogen atoms in the minimum ring framework path of a ring defined by the covalent organic framework.

17. The covalent organic framework composition of claim 15, wherein the minimum ring framework path of a ring defined by the covalent organic framework is substantially free of imines.

18. The covalent organic framework composition of claim 15, wherein the minimum ring framework path of a ring defined by the covalent organic framework comprises 52 heavy atoms to 170 heavy atoms, or wherein the minimum ring framework path of a ring defined by the covalent organic framework comprises 78 heavy atoms to 210 heavy atoms.

19. A method of sorbing CO2, comprising exposing a composition according to claim 15 to a gas phase environment comprising CO2.

20. The method of adsorbing CO2 of claim 19, wherein the composition is exposed to a gas phase environment comprising 6.0 vol % or less of CO2, or wherein the composition is exposed to a gas phase environment comprising 8.0 vol % to 20 vol % of CO2.
